(19) United States
(12) Patent Application Publication
(10) Pub. No.: US 2012/0318260 A1
(43) Pub. Date: Dec. 20, 2012

(54) PORTABLE NEBULIZING APPARATUS

(75) Inventors: SHU-PIN HSIEH, TAOYUAN COUNTY (TW); YU-JU SU, TAOYUAN COUNTY (TW); CHIA-CHEN HUANG, TAOYUAN COUNTY (TW); CHIEN-HUA LIN, TAOYUAN COUNTY (TW); YU-CHUNG HSU, TAOYUAN COUNTY (TW); TAI-SHUAN LIN, TAOYUAN COUNTY (TW); MEI-HUI HUANG, TAOYUAN COUNTY (TW); CHIEH-MING HSIUNG, TAOYUAN COUNTY (TW)

(73) Assignee: MICROBASE TECHNOLOGY CORP., TAOYUAN COUNTY (TW)

(21) Appl. No.: 13/235,591

(22) Filed: Sep. 19, 2011

(30) Foreign Application Priority Data
Jun. 14, 2011 (TW) 100210744

(51) Int. Cl. A61M 11/02

(52) U.S. Cl. 128/200.23

(57) ABSTRACT

A nebulizing apparatus includes a housing, a carrying cavity, an nebulizing element, a slide element and a circuit board. The carrying cavity is formed in the housing for carrying a nebulized liquid and the carrying cavity includes a through hole. The nebulizing element is installed at the through hole. The slide element is slidably installed on a side of the housing and includes a magnet. The circuit board is installed on an internal side of the housing and includes a magnetic switch installed on a side of the circuit board. When the slide element is move to an ON position, the magnetic switch is turned on by the magnetism of the magnet, so that the nebulizing element can spray the nebulized liquid.

### CROSS-REFERENCE TO RELATED APPLICATIONS

This non-provisional application claims priority under 35 U.S.C. §119(a) on Patent Application No(s). 100210744 filed in Taiwan, R.O.C. on Jun. 14, 2011, the entire contents of which are hereby incorporated by reference.

### BACKGROUND OF THE INVENTION

1. Field of the Invention

The present invention relates to a nebulizing apparatus, in particular to a portable nebulizing apparatus.

2. Description of the Related Art

As the demand for air fresheners and pharmaceutical sprays increases day after day, the use of nebulizer for nebulizing an aromatic solutions and pharmaceutical solutions also increases correspondingly. Aerosol sprays are further used for moisturizing indoor air in districts or countries of a relatively dry weather, in addition to its uses as the air freshener and pharmaceutical spray.

Conventional nebulizers are generally divided into two main types: a nozzle type nebulizer and a piezoelectric nebulizer. Most of the nozzle type nebulizers pressurize a liquid and spray the liquid through a nozzle. When the compressed liquid flows through an exit of the nozzle, the width of the exit becomes wider, and the instantaneous external pressure value is less than the saturated vapor pressure of the liquid, so that the liquid can be evaporated to form a mist. Although the nozzle type nebulizer has the features of a simple structure and a low cost, yet the mist particles are non-uniform, and adjacent mist particles may be combined to produce droplets easily, thus causing problems to the use of the nebulizer. On the other hand, the piezoelectric nebulizer uses a high-frequency oscillating device (such as a piezoelectric driving component) which is submerged in the liquid, such that the energy produced by the oscillation of the oscillating device can break the surface tension of the liquid to molecularize the liquid or a mircoporous nebulizing plate installed on a side of an oscillator or inside the oscillator is adopted, such that the microporous nebulizing plate can molecularize the liquid by excitations produced by the wavy kinetic energy to achieve the nebulization effect. The piezoelectric nebulizer has the advantage of producing uniform mist particles, but it incurs a higher cost, and thus the piezoelectric nebulizer is generally used in an application that requires a high precision spray only, but will not be used extensively in the mass market.

As the piezoelectric spray technology advances, related key components become increasingly smaller and the manufacturing cost drops drastically. Since the piezoelectric nebulizer can spray uniform and tiny mist particles (up to a nanoscale), the piezoelectric nebulizer used as a device for spraying pharmaceutical solution improves the absorbing effect by patients and becomes a portable nebulizing apparatus that gradually replaces the conventional nozzle type nebulizer.

### SUMMARY OF THE INVENTION

Therefore, it is a primary objective of the present invention to provide a portable nebulizing apparatus for moisturizing or freshening surrounding air.

To achieve the foregoing objective, the present invention provides a portable nebulizing apparatus, comprising a housing, a carrying cavity, an nebulizing element, a slide element and a circuit board. The carrying cavity is formed in the housing for carrying a nublized liquid and the carrying cavity includes a through hole. The nebulizing element is installed at the through hole. The slide element is slidably installed on a side of the housing and includes a magnet. The circuit board is installed on an internal side of the housing and includes a magnetic switch installed on a side of the circuit board.

Wherein, the circuit board further includes a light emitting diode (LED) installed on a side of the circuit board.

Wherein, the slide element includes a slide cover and a slide cover switch component, and the slide cover switch component is installed between the slide cover and the circuit board, and the slide cover includes a transparent light shade installed thereon and corresponding to the LED.

Wherein, the slide cover switch component is a stepless component.

Wherein, the portable nebulizing apparatus further includes an upper casing and a pressure absorbing element, and the upper casing is covered onto an end of the housing, and the pressure absorbing element is installed between the carrying cavity and the upper casing.

Wherein, the nebulizing element includes a piezoelectric plate and a nozzle plate, and the nozzle plate is installed between the piezoelectric plate and the through hole.

Wherein, the nebulizing element further includes a nozzle plate cover for positioning the piezoelectric plate and the nozzle plate to a side of the through hole.

Wherein, the nozzle plate includes a plurality of firing holes.

Wherein, the nozzle plate further includes a plurality of first protrusions substantially in a non-circular shape, and installed on the nozzle plate and arranged with an interval from the center of the nozzle plate, wherein the first protrusions are arranged in a geometric pattern by using the center of the nozzle plate as a basis.

Wherein, the portable nebulizing apparatus further comprises a battery base and a lower casing, and the battery base is disposed on an internal side of the housing, and the lower casing is covered onto another end of the housing.

Wherein, the magnetic switch is a reed switch.

### BRIEF DESCRIPTION OF THE DRAWINGS

FIG. 1 is an exploded view of a portable nebulizing apparatus in accordance with a preferred embodiment of the present invention;

FIG. 2 is a perspective view of a portable nebulizing apparatus in accordance with a preferred embodiment of the present invention;

FIG. 3 is a schematic view of a portable nebulizing apparatus in accordance with a first preferred embodiment of the present invention;

FIG. 4 is a schematic view of a nozzle plate of a portable nebulizing apparatus in accordance with a second preferred embodiment of the present invention;

FIG. 5 is a perspective view of a portable nebulizing apparatus situated at an OFF position in accordance with a preferred embodiment of the present invention;

FIG. 6 is a perspective view of a portable nebulizing apparatus situated at an OFF position in accordance with a preferred embodiment of the present invention;

FIG. 7 is a perspective view of a portable nebulizing apparatus situated at an ON position in accordance with a preferred embodiment of the present invention; and

FIG. 8 is a cross-sectional view of a portable nebulizing apparatus situated at an ON position in accordance with a preferred embodiment of the present invention.

### DETAILED DESCRIPTION OF THE PREFERRED EMBODIMENTS

The technical content of the present invention will become apparent by the detailed description of the following embodiments and the illustration of related drawings as follows.

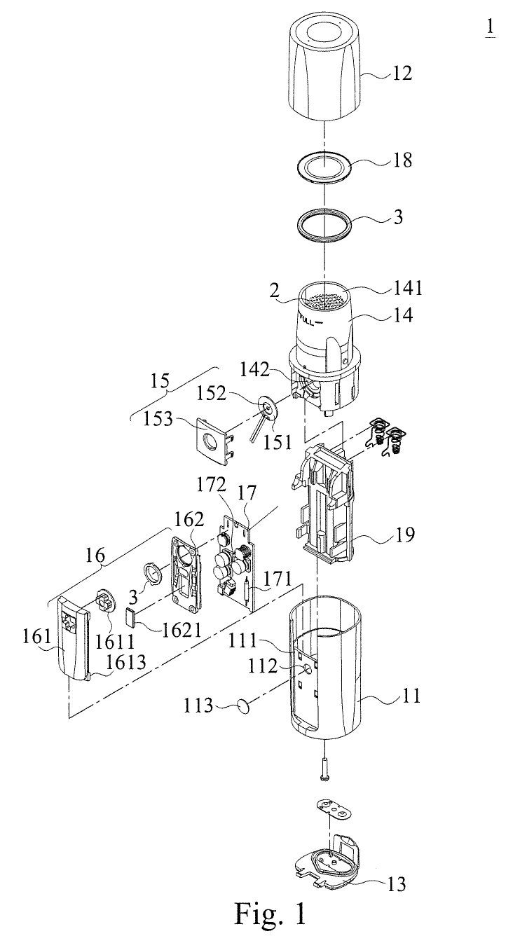

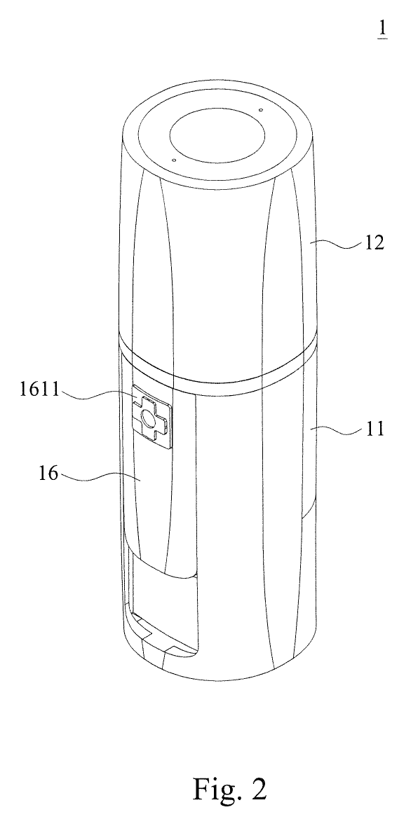

With reference to FIGS. 1 and 2 for an exploded view and a perspective view of a portable nebulizing apparatus in accordance with a preferred embodiment of the present invention, the portable nebulizing apparatus 1 comprises a housing 11, an upper casing 12, a lower casing 13, a carrying cavity 14, an nebulizing element 15, a slide element 16, a circuit board 17, a pressure absorbing element 18 and a battery base 19.

The housing 11 is made of plastic or metal. In this preferred embodiment, the housing 11 is substantially in a cylindrical shape, and includes a plurality of guide rails 111 and a hole 112 formed at a lateral edge of the housing 11 and corresponding to the slide element 16, and the hole 112 is covered by cover 113.

The upper casing 12 is installed on a side of the housing 11, and the lower casing 13 is installed to another side of the housing 11. In this preferred embodiment, the upper casing 12 and the lower casing 13 are made of the same material of the housing 11 which can be plastic or metal. The housing 11, the upper casing 12 and the lower casing 13 are assembled into a stylish shape including but not limited to a lipstick.

The carrying cavity 14 is formed on a side of the housing 11, and includes a liquid carrying chamber 141, a through hole 142 and a latch portion 143. The liquid carrying chamber 141 is provided for carrying a nebulized liquid 2. The through hole 142 is formed corresponding to the nebulizing element 15. In this preferred embodiment, the nebulized liquid 2 is a pharmaceutical spray, an aromatic essential oil or pure water for moisturizing air. The latch portion 143 is latched at an end of the housing 11.

The nebulizing element 15 includes a piezoelectric plate 151, a nozzle plate 152 and a nozzle plate cover 153. The nozzle plate 152 is installed between the piezoelectric plate 151 and the through hole 142, and the nozzle plate cover 153 is provided for covering and positioning the piezoelectric plate 151 and the nozzle plate 152 to a side of the through hole 142.

The slide element 16 is installed outside a lateral edge of the housing 11 and includes a slide cover 161 and a slide cover switch component 162. The slide cover switch component 162 is installed on a side of the slide cover 161. Wherein, the slide cover 161 includes a transparent light shade 1611 and a plurality of ribs 1613. Wherein, the slide cover 161 is coupled to the guide rails 111 by the ribs 1613 and slid along the lateral edge of the housing 11 by a stepless way to an ON position or an OFF position. Wherein, the slide cover switch component 162 further includes a magnet 1621 installed thereon. In this preferred embodiment, the transparent light shade 1611 can form a cross-shaped decorative pattern, but the pattern formed by the invention is not limited to this shape only.

The circuit board 17 is installed in the housing 11. Wherein, the circuit board 17 includes a magnetic switch 171 and an LED 172 installed at positions corresponding to a side of the slide element 16. In this preferred embodiment, the magnetic switch 171 is a reed switch, and the LED 172 is installed at a position corresponding to the hole.

The pressure absorbing element 18 is installed between the upper casing 12 and the carrying cavity 14. The pressure absorbing element 18 is provided for preventing the situation of having difficulty to open the upper casing 12 due to a negative pressure inside the carrying cavity 14 after the upper casing 120 seals the carrying cavity 14.

The battery base 19 is installed in the housing 11, and the circuit board 17 is latched onto a side of the battery base 19. The battery base 19 is provided for installing a battery to supply electric power to the circuit board 17. In addition, the piezoelectric plate 151 is electrically coupled to the circuit board 17.

To prevent a leakage of the nebulized liquid 2 stored in the carrying cavity 14, a waterproof ring 3 is installed separately between the upper casing 12 and the carrying cavity 14, and between the slide cover switch component 162 and the nozzle plate cover 153.

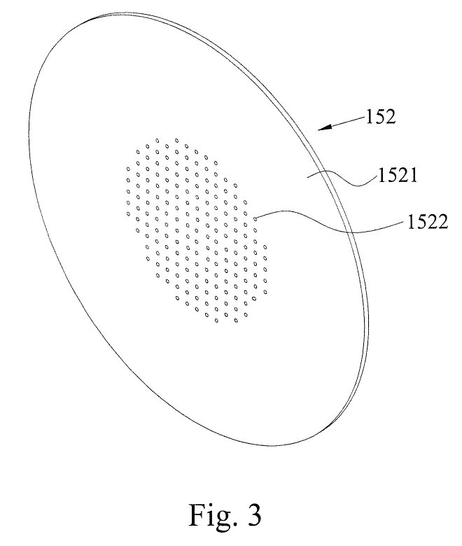

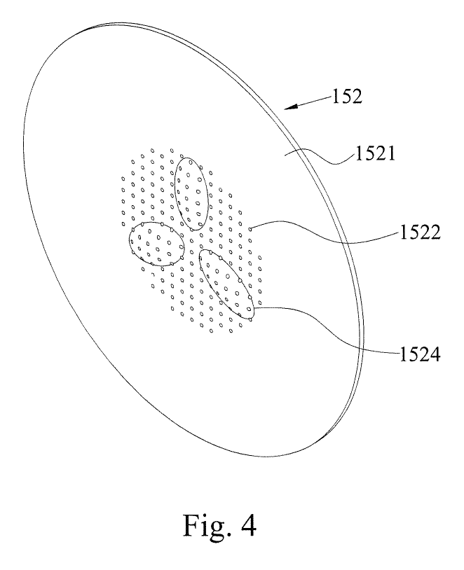

With reference to FIGS. 3 and 4 for the schematic views of a nozzle plate of a portable nebulizing apparatus in accordance with the first and second preferred embodiments of the present invention respectively, the nozzle plate 152 of these preferred embodiments is a simple nozzle plate comprising a circular plate 1521 with a plurality of circular firing holes 1522 formed thereon, or an enhanced nozzle plate comprising a circular plate 1521, a plurality of first protrusions 1524 and a plurality of circular firing holes 1522 for enhancing the nebulizing effect and the manufacturing process. Wherein, the first protrusions 1524 are substantially in a non-circular shape and installed on the nozzle plate 152, and arranged with an interval apart from the center of the nozzle plate 152, and the center of the nozzle plate 152 is used as a basis for forming a geometric pattern, such that the nozzle plate 152 has a non-cambered cross-section to achieve the effects of enhancing the stability of the nebulizing process and the nebulizing quantity, reducing the difficulty of the manufacture, and strengthening the low frequency resonance resistance. In this preferred embodiment, the shape of the first protrusions 1524 is not limited to that given in the figure, but the first protrusions 1524 can be in a rhombus shape, elliptical shape, triangular shape, hourglass shape, meniscus shape or any other shape; and the first protrusions 1524 has a cross-section which is a triangular cone, a single-arc plane, an oblique plane, a trapezium plane, or any other form.

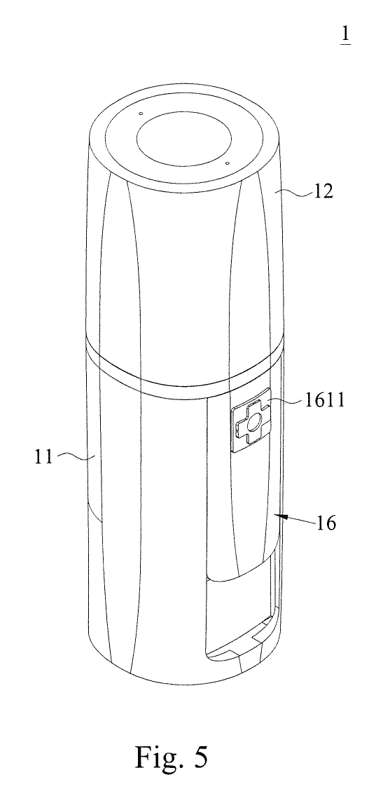

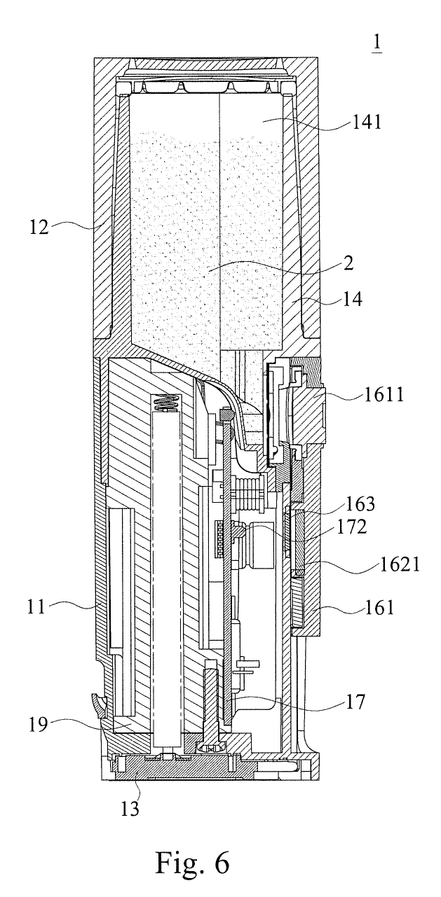

With reference to FIGS. 5 and 6 for a perspective view and a cross-sectional view of a portable nebulizing apparatus situated in an OFF position in accordance with the present invention respectively, when the slide element 16 is moved to an OFF position, the slide element 16 covers the nozzle plate cover 153. Now, the magnet 1621 of the slide cover switch component 162 is situated at a position away from the magnetic switch 171 of the circuit board 17, and the magnetic switch 171 disconnects the nozzle plate 152 and the circuit board 17, such that the nebulizing element 15 is idle. When the slide element 16 is pushed and moved to an ON position, the nozzle plate cover 153 is not covered by the slide element 16 but it is exposed, and the magnet 1621 is near the magnetic switch 171, so that the magnetic switch 171 is induced to electrically connect the piezoelectric plate 151 and the circuit board 17. After the piezoelectric plate 151 is electrically conducted, its oscillation can drive the nozzle plate 152 to vibrate, and a liquid near the circular firing hole 1522 of the nozzle plate 152 can be divided into smaller molecules by the action of Rayleigh waves to facilitate the spray and nebulization of the liquid.

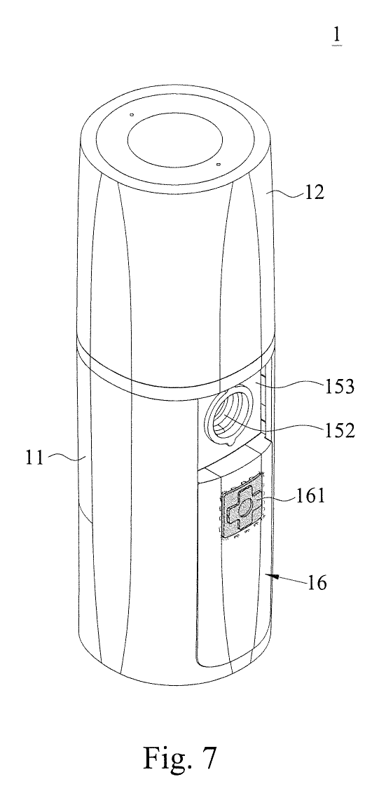

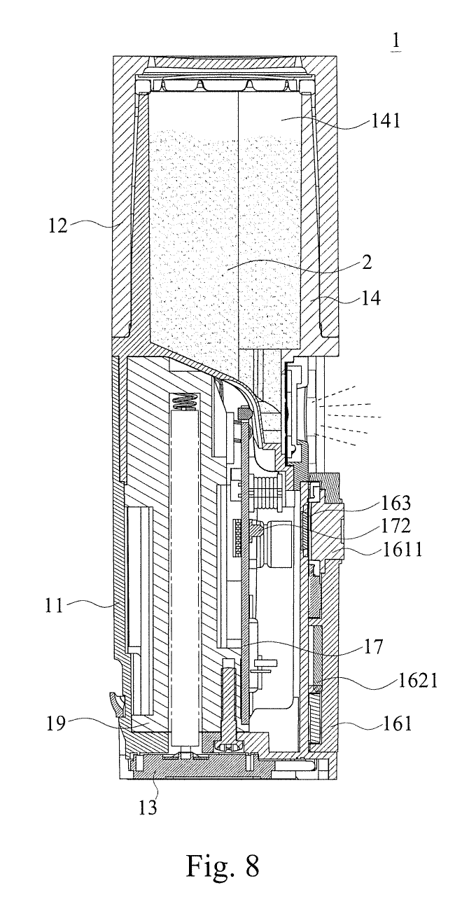

With reference to FIGS. 7 and 8 for a perspective view and a cross-sectional view of a portable nebulizing apparatus situated in an ON position in accordance with the present invention respectively, when the slide element 16 is moved to the ON position, the transparent light shade 1611 is situated precisely to the LED 172 and hole 162 on the circuit board, and a portion of the light emitted from the LED 172 passes through the hole 112, the cover 113 and the transparent light shade 1611 to the outside.

In summation, the portable nebulizing apparatus of the present invention has the advantages of a light weight and an easy carry, and adopts a piezoelectric nebulizing plate to provide a more stable and uniform nebulizing effect than the conventional nozzle type nebulizers. Obviously, the invention improves over the prior art and complies with the patent application requirements, and thus is duly filed for patent application.

While the invention has been described by means of specific embodiments, numerous modifications and variations could be made thereto by those skilled in the art without departing from the scope and spirit of the invention set forth in the claims.

What is claimed is:

1. A portable nebulizing apparatus, comprising:
a housing;
a carrying cavity, formed inside the housing, and having a through hole, and provided for carrying a nebulized liquid;
an nebulizing element, installed at the through hole;
a slide element, slidably installed on a side of the housing, and having a magnet installed on the slide element; and
a circuit board, installed on an internal side of the housing, and having a magnetic switch installed on a side of the circuit board.

2. The portable nebulizing apparatus of claim 1, wherein the circuit board further includes a light emitting diode (LED) installed on a side of the circuit board.

3. The portable nebulizing apparatus of claim 2, wherein the slide element includes a slide cover and a slide cover switch component, and the slide cover switch component is installed between the slide cover and the circuit board, and the slide cover includes a transparent light shade installed thereon and corresponding to the LED.

4. The portable nebulizing apparatus of claim 3, wherein the slide cover switch component is a stepless component.

5. The portable nebulizing apparatus of claim 1, further comprising an upper casing and a pressure absorbing element, and the upper casing is covered onto an end of the housing, and the pressure absorbing element is installed between the carrying cavity and the upper casing.

6. The portable nebulizing apparatus of claim 1, wherein the nebulizing element includes a piezoelectric plate and a nozzle plate, and the nozzle plate is installed between the piezoelectric plate and the through hole.

7. The portable nebulizing apparatus of claim 6, wherein the nebulizing element further includes a nozzle plate cover for positioning the piezoelectric plate and the nozzle plate to a side of the through hole.

8. The portable nebulizing apparatus of claim 6, wherein the nozzle plate includes a plurality of firing holes.

9. The portable nebulizing apparatus of claim 8, wherein the nozzle plate further includes a plurality of first protrusions substantially in a non-circular shape, and installed on the nozzle plate and arranged with an interval from the center of the nozzle plate, and in a geometric pattern by using the center of the nozzle plate as a basis.

10. The portable nebulizing apparatus of claim 1, further comprising a battery base and a lower casing, and the battery base being disposed on an internal side of the housing, and the lower casing being covered onto another end of the housing.

11. The portable nebulizing apparatus of claim 1, wherein the magnetic switch is a reed switch.
